(19) United States
(12) Patent Application Publication
(10) Pub. No.: US 2026/0345941 A1
(43) Pub. Date: Nov. 5, 2026

(54) Belt Webbing System for Sleep Apnea/ CPAP Machine

(71) Applicant: Vishnu Vardhan Chakravaram, Gallatin, TN (US)

(72) Inventor: Vishnu Vardhan Chakravaram, Gallatin, TN (US)

(73) Assignee: Vishnu Vardhan Chakravaram, Gallatin, TN (US)

(21) Appl. No.: 19/196,562

(22) Filed: May 1, 2025

(51) Int. Cl. A61M 16/08; A61M 25/02

(52) U.S. Cl. CPC A61M 16/0875; A61M 2025/0206; A61M 2025/0213; A61M 2025/026; A61M 2210/0618

(57) ABSTRACT

A belt webbing system that goes around torso of an individual. Belt webbing system holds the hose of the CPAP machine at the user's desired location and thus reduces the amount of pull hose exerts on the nose, due to the mass of the hose. The belt webbing system provides a convenience to the user by holding the hose, and the possibility to slide the hose from one position on the webbing to another position. In the absence of such an arrangement, the nose becomes sore. And when the individual in sleep turns left or right, hose's nasal pillow may move from its position causing air to leak.

### CROSS-REFERENCE TO RELATED APPLICATIONS

This application takes the benefit of provisional application 63/558,827 filed on Feb. 28, 2024.

### BACKGROUND OF THE INVENTION

### (1) Field of the Invention

When an individual with sleep apnea wears a nasal pillow which is connected to the hose, he or she experiences pull or mass of the hose on the nose and it causes great amount of inconvenience in terms of soreness in the nose after walking up & leakage in pressurized air. Belt webbing system for CPAP machine is a belt system to hold hose of CPAP machine at a desired location on the belt. This device/invention helps the user by reducing or preventing and taking on weight and pull of the hose on users nose. This device is configured in such a way that the hose can slide across the belt webbing as per the users need and/or convince.

(2) Individual with sleep apnea depend on cpap machine to maintain adequate blood oxygen levels, if the level of oxygen in blood is not sufficient it may cause lots of long-term health issues. Often in the middle of the night nose pillow slips away from the nose causing the sealed connection between nose and cpap machine to compromise. If such a compromise happens the purpose of cpap machine is not survived. One of the most probable reasons for such a compromise is mass and pull of hose. Belt webbing system's aim is to isolate or prevent this issue by taking the weight and pull of the hose onto itself and relieve the nose. Because of this arrangement integrity of sealed connection between the nose and the CPAP machine is preserved/sustained.

### BRIEF SUMMARY OF THE INVENTION

In one embodiment the composite structure is a belt webbing system wherein the belt webbing system comprises of a belt webbing, a rectangular plate with two slots wherein each slot has a central bar located at the center, a circular hole at the center of the rectangular plate. The composite structure further comprises of two-piece quick release buckle, Velcro and a Revit.

To illustrate how to put together previously mentioned parts into a belt webbing system. One end of the Belt webbing is weaved through slots and their respective central slots of the rectangular plate and is pulled out of the second slot. The end of belt webbing that was weaved through the rectangular plate is weaved around the rib of male quick release buckle and this belt webbing end is stitched to itself. The other end of the belt webbing goes around the rib of the female quick release buckle and is stitched to itself. To the central hole of rectangular plate using a Revit, Velcro is riveted. However, Velcro can go around its own axis. And rectangle plate can slide across the length of the belt webbing.

To illustrate the usage of the composite structure, first the user routes belt webbing around the torso and connects the male and female two-piece quick release buckle. Next step user places hose of CPAP machine on the Velcro riveted to rectangular plate at a desired location and secures the location with Velcro. User then wears the nasal pillow and secures its position with a face strap that goes around the head. The arrangement around the head and nasal pillow can be of various configurations. User then slides the rectangle place across the belt webbing based on the side user intends to sleep. The diameter of the belt webbing system can be adjusted by the user at the two-piece quick release buckle.

### BRIEF DESCRIPTION OF THE SEVERAL VIEWS OF THE DRAWING(S)

FIG. 1 is an illustrative isometric view of the rectangle plate of the present invention in use.

FIG. 2 is an illustrative isometric view of a two-piece quick release buckle of present invention in use.

FIG. 3 is an illustrative isometric view of belt webbing of present invention in use.

FIG. 4 is an illustrative isometric view of Velcro of present invention in use.

FIG. 5 is an illustrative isometric view of Revit of present invention in use.

FIG. 6 is an illustrative isometric view of the belt webbing system of present invention in use.

FIG. 7 an illustrative isometric view of bedroom setup with an individual using the webbing belt system with cpap machine and hose.

### DETAILED DESCRIPTION OF THE INVENTION

The invention will be primarily described within the context of embodiments; however, those skilled in art and informed by the teachings herein will realize that the invention is also applicable to other technical areas and/or embodiments. Practitioners skilled in the art will recognize numerous other embodiments as well.

The illustrative embodiments described herein are not meant to be limiting. It may be readily understood that certain aspects of the disclosed embodiments can be arranged and combined in a variety of different configurations, all of which are contemplated herein.

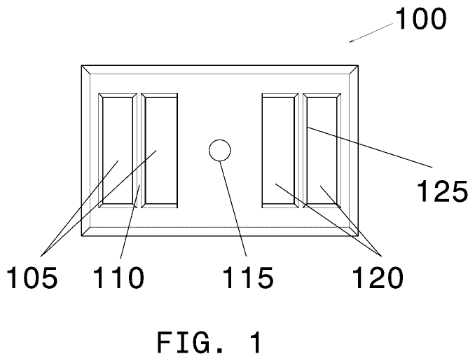

FIGS. 1-7 illustrates a composite structure 100, 200, 300, 400, 500, 600, 700.

Referring to FIG. 1 depicts an front view of rectangle plate 100, which has two identical slots 105 & 120, each of these slots contain identical central bars 110 & 125, and at the center of the rectangular plate 100 a central hole 115 is located.

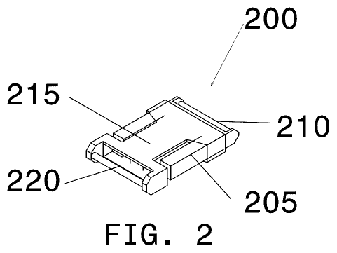

Referring to FIG. 2 depicts an isometric view of two-piece quick release buckle 200, which contains a male and female buckle 205 & 215, respectively. When the male and female buckles are plugged into each other, a firm connection is made until the user intends to disconnect. Each of the buckles 205 & 215 has a rib 210 & 220. Ribs 210 & 220 run across breadth.

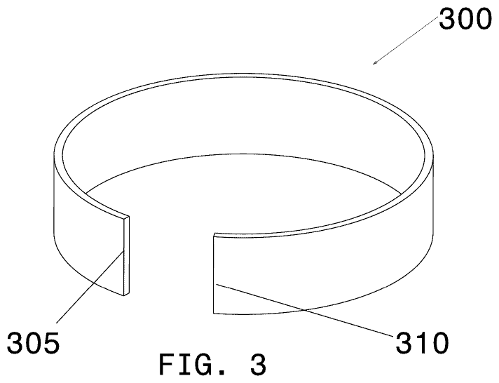

Referring to FIG. 3 depicts an isometric view of belt webbing 300. One end of belt webbing 305 is weaved through slots 105 & 120 of rectangle plate 100 from FIG. 1 and their central ribs 110 & 125, respectively. Belt webbing end 305 goes around the rib 210 of male buckle 205 and the belt webbing end 305 after going around rib 210 is stitched to belt webbing 300. The other end of belt webbing 310 goes around the female buckle 215's rib 220. After the belt webbing end 310 goes around the rib 220, belt webbing end 310 is stitched to 300.

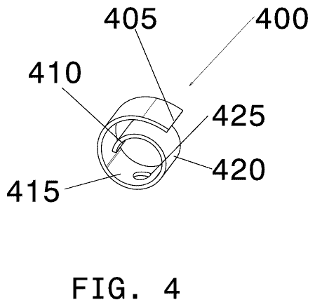

Referring to FIG. 4 depicts an isometric view of Velcro 400 which is of considerable length and has two ends 405 & 410. Velcro 400 also has two sides 415 top & 420 bottom. A hole 425 is located on 400 at a certain distance from the 410 side. Hole 424 passes through sides 415 & 420. The sides 415 & 420 are designed to make and keep a firm contact if placed on each other.

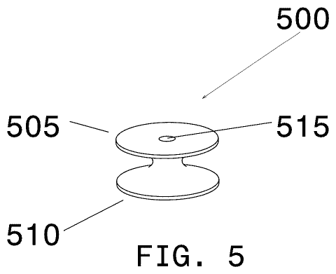

Referring to FIG. 5, depicts an isometric view of Revit 500 which is of two parts 505 & 510 with hole in the center 515. This Revit 500 is used to hold Velcro 400 from FIG. 4 and rectangle plate 100 from FIG. 1 together. Velcro 400 after riveted to rectangle plate 100 can be able to go around its own axis.

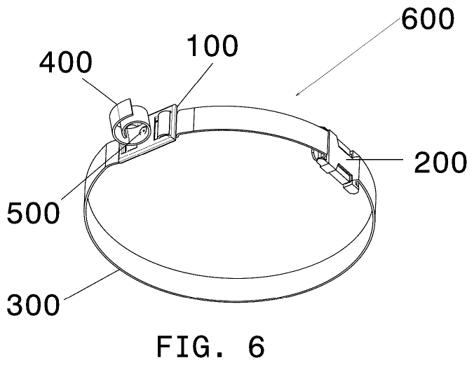

Referring to FIG. 6 depicts an isometric view of composite structure belt webbing system 600, which preferably consists of 5 segments namely (1) rectangle plate 100 (2) two-piece quick release buckle 200, (3) belt webbing 300, (4) Velcro 400, (5) Revit 500. The belt webbing system 600 wherein belt webbing 300 is woven through rectangle plate 100-and two-piece quick release buckle 200 is used to connect the two ends of belt webbing 300. The Velcro 400 and rectangle plate 100 are held to each other using a Revit 500. Velcro 400 can go around its own axis even after riveting to rectangle plate 100, using Revit 500. The rectangle plate 100 can slide across belt webbing 300.

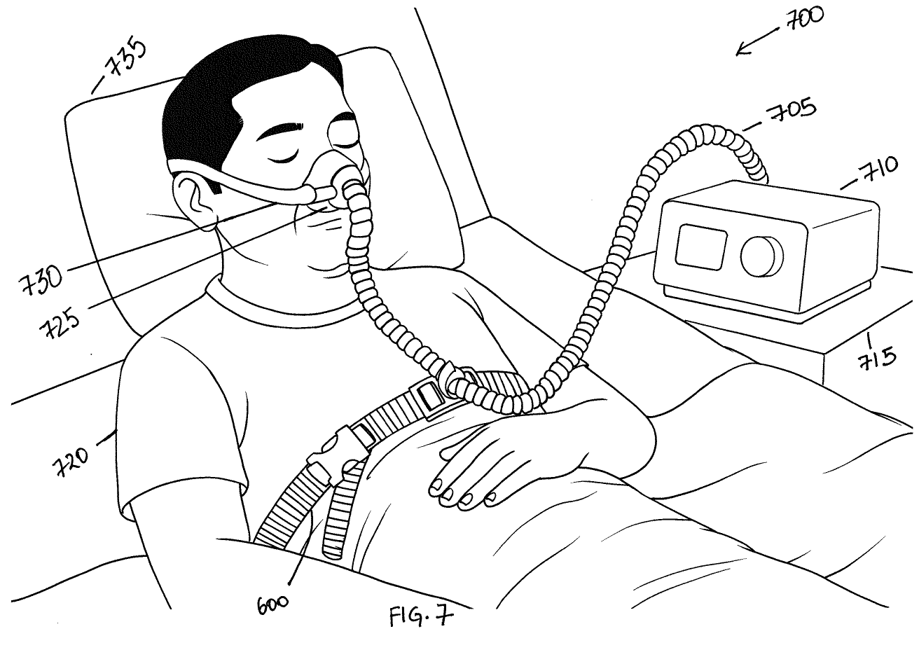

Referring to FIG. 7 depicts an isometric view of human on his back lying on a bed with belt webbing system in real time use scenario 700, which preferably consists of 8 segments namely (1) Hose 705, (2) CPAP machine 710, (3) Night Stand 715, (4) Belt webbing system 600, (5) human 720, (6) Nasal pillow 725,(7) face strap 730, (8) pillow 735.

1. A webbing belt system, the system comprising:
A webbing belt of considerable length;
A rectangle plate with a flat profile made from flexible or strong material; wherein the rectangle plate comprises:
a couple of slots that are positioned parallel to each other lengthwise; each slot contains a central bar; the central bar guides belt webbing;
a central hole is in the middle of the said rectangle plate; the rectangle plate has raised perimeter to provide strength; the said webbing belt is threaded through the slot around said central bar on both the slots; the said rectangle plate can slide across the webbing belt;
a Velcro is riveted to the central hole at one third length of the Velcro from one of the sides;
a hose whose one end connected to cpap machine and other end to nasal pillow is placed on the Velcro exactly on the central hole; Velcro is wrapped around the hose; this arrangement holds the hose in place;
a two-piece quick release buckle made out of flexible material: wherein the two-piece quick release buckle comprises:
a male end that has a slot around which first end of the webbing belt is guided and is stitched to itself to form a closed loop around the slot;
a female end of two-piece buckle system has a slot around which second end of the webbing belt is guided and can loosened or tightened at this end.

2. The webbing belt system according to claim 1, wherein the webbing belt system is worn by the user around the torso.

3. The webbing belt system according to claim 1, wherein the Velcro locking the hose at a desired place prevents weight of the hose exerted on the nose of the user.

4. The webbing belt system according to claim 1, wherein the webbing belt system bears the pull of hose between webbing belt system and cpap machine.

5. The webbing belt system according to claim 1, wherein the user can adjust position of the rectangle plate by sliding it across the webbing belt.

6. The webbing belt system according to claim 1, wherein the Velcro can rotate around its own axis that overlaps with the central hole and Revit.

7. The webbing belt system according to claim 6, wherein the user does not have to slide the rectangle plate which holds the hose to be in line with the user's nose.

8. The webbing belt system according to claim 1, air will not leak between nostrils and nose pillow because weight of the hose is on the webbing belt system.

9. The webbing belt system according to claim 1, diameter of belt webbing can be adjusted at the two-piece quick release buckle.

10. The webbing belt system according to claim 1, hose can be locked in place gently meaning hose can slide through Velcro lock.

11. The webbing belt system according to claim 1, wherein hose is locked in place meaning hose cannot slide though the Velcro lock.
